(19) United States
(12) Patent Application Publication
(10) Pub. No.: US 2022/0399105 A1
(43) Pub. Date: Dec. 15, 2022

(54) MONITORING AND ENFORCING INFECTION SAFETY PROCEDURES IN OPERATING ROOMS

(71) Applicant: OnSite-LLC, Indianapolis, IN (US)

(72) Inventors: Jennifer A. Wagner Block, Discovery Bay, CA (US); Damon G. Greeley, Fort Mill, SC (US)

(73) Assignee: OnSite-LLC, Indianapolis, IN (US)

(21) Appl. No.: 17/776,951

(22) PCT Filed: Nov. 13, 2020
(86) PCT No.: PCT/US20/60608

§ 371 Date: May 13, 2022

Related U.S. Application Data
(60) Provisional application No. 62/935,951, filed on Nov. 15, 2019.

(51) Int. Cl. G16H 40/20; G16H 10/60; G08B 21/24; G06V 10/28; G06V 20/64; G06V 40/16

(52) U.S. Cl. CPC G16H 40/20; G16H 10/60; G08B 21/245; G06V 10/28; G06V 20/64; G06V 40/161

(57) ABSTRACT

An operating room monitoring system (ORMS) may be uniquely configured for a particular operating room in order to provide custom monitoring and responses based on the unique characteristics of that operating room. The ORMS includes a sensor array of individual sensors placed around the operating room in positions determined by the type of sensor and the unique characteristics of the operating room. Data from the sensor array is collected and analyzed using an infection-reduction protocol. Analyzed data may include images and video, proximity data, airflow data, and climate data, for example. Protocol violations may result in tailored responses such as automatic adjustment of HVAC equipment, or generalized responses such as room-level visual or audible alerts. A dashboard interface may be displayed on various devices that provides sensor data, environmental quality data, risk picturing, and other information usable to mitigate infection risks.

### REFERENCE TO RELATED APPLICATION

This application is a national stage application, filed under 35 U.S.C. 371, of International Application No. PCT/US2020/060608, filed on Nov. 13, 2020, having title “Monitoring and Enforcing Infection Safety Procedures in Operating Rooms,” which claims priority to, and benefit of U.S. Provisional Patent Application No. 62/935,951, filed on Nov. 15, 2019, having title “Monitoring and Enforcing Infection Safety Procedures in Operating Rooms,” the whole of the document being incorporated herein by reference.

### FIELD

The present disclosure relates to a sensor field and context camera-based compliance system for an operating room which actively monitors a defined space and detects various movements or conditions that violate a predefined optimized plan for the given operating room to reduce the risk of patient infection and/or other adverse events.

### BACKGROUND

The health care industry is consistently faced with the challenge of improving the quality of care while simultaneously reducing costs. A recent study reported that the health care industry sustains $10 billion in annual costs related to infections acquired after admission. See Zimlichman E., Health Care Associated Infections a Meta-Analysis of Costs and Financial Impacts on the US Health Care System. JAMA Intern Med 12013; 173:2039-46. Similarly, the Center for Disease Control and Prevention reported that 1 in 20 patients admitted to hospitals will contract a hospital-acquired infection. Medical insurance companies and government payers have taken note of these costs and are reducing reimbursement for health care-associated infections. These include surgical site infections, which can be impacted by the quality of the air environment in the operating room and the compliance with proper procedures, guidelines, and optimal plans by the hospital staff during surgery.

Potentially high-risk medical procedures are performed in hospital operating rooms across the country on a daily basis. Surgical sites can be infected by entrainment of harmful organisms coming from the contaminated air zone by both airborne and contact transmission modes. Common causes include work done in contaminated air zones, errors in compliance with sterile techniques, fan-cooled equipment back pressure on room counter flow, and relative humidity in the environment.

As a result, there are detailed and stringent procedures in place for routine clinical practices, such as scrubbing and gowning requirements, gloving, masking, and instrument sterilization. Many other procedures and requirements govern the HVAC (heating, ventilation, and air conditioning) systems for operating rooms, such as relative humidity and ventilation rates. To provide a safe environment for surgery, ventilation rates in operating rooms, which are measured in air changes per hour (ACH), are understandably higher than any other space in a hospital.

Studies have shown that ACH rates and other controls exceeding those provided by guidelines and/or regulations do not successfully improve contamination control. While these elevated air change rates and other procedures may be required to provide a quality indoor environment to help minimize the risk of surgical site infections, there are significant capital and operating costs associated with meeting these requirements. Excess costs are often incurred by hospitals seeking to exceed these guidelines with very little improvement realized. Moreover, the guidelines are generally applicable to each and every operating room and do not allow for any variability based upon the actual layout of the operating room and utilization, which may be unique to each case or surgical technique type.

What is needed is an improved system and approach to operating room procedures.

### BRIEF DESCRIPTION OF THE DRAWINGS

FIG. 1 is a schematic diagram of an exemplary operating room monitoring system;

FIG. 2 is a schematic diagram of an exemplary operating room including the operating room monitoring system of FIG. 1;

FIG. 3 is a schematic diagram illustrating risk areas in the operating room of FIG. 2;

FIG. 4 is a schematic diagram illustrating personnel areas in the operating room of FIG. 2;

FIG. 5 is a schematic diagram illustrating different air quality zones in the operating room of FIG. 2;

FIG. 6 is a screenshot of an exemplary interface that may be displayed to provide a risk picture for the operating room of FIG. 2;

FIG. 7 is a screenshot of an exemplary interface that may be displayed to provide an environmental quality dashboard displaying information associated with the operating room of FIG. 2;

FIG. 8 is a flowchart of an exemplary set of steps that may be performed with the operating room monitoring system of FIG. 1; and

FIG. 9 is a flowchart of an additional exemplary set of steps that may be performed with the operating room monitoring system of FIG. 1.

### DETAILED DESCRIPTION

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

While present operating room guidelines can be effective in reducing infection rates, the availability of further improvements in operating room practices and procedures have been studied by the present inventors. Specifically, in Methodology for Analyzing Environmental Quality Indicators in a Dynamic Operating Room Environment. Am. J. of Infection Control; 45 (2017) 354-359, which is incorporated by reference herein, it was identified that any unique operating room could be analyzed using a standard environmental analysis protocol to develop guidance for operational practices and regulatory requirements to improve patient outcomes, and specifically to lower infection rates.

These experiments included over 20,000 hours of work and the analysis of over 100,000 unique data points. Testing was done using multiple pieces of equipment including velocity and temperature sensors, particle counters, and bacterial assessment tools. Numerous mock procedures were performed while measuring critical environmental indicators with portable particle and microbiological air sampling equipment, video and thermal cameras, scripted movements, inert smoke machines, and temperature, air velocity, and room humidity sensors. The levels of microbial contamination and determination of the specific microorganisms gathered within the environmental biome are the sources of those found in surgical site infections (“SSIs”).

Knowledge of how to best modify and utilize a room to minimize bacterial contamination of the patient has been applied to twenty operating rooms. Over a study period of 4 years, a significant drop in SSI rates was discovered and determined to be statistically significant compared to a control set of other unmodified operating rooms that had relatively no change in SSI rates. The present disclosure describes a system and method that may be implemented to monitor and enforce improved operating room guidelines such as those described above.

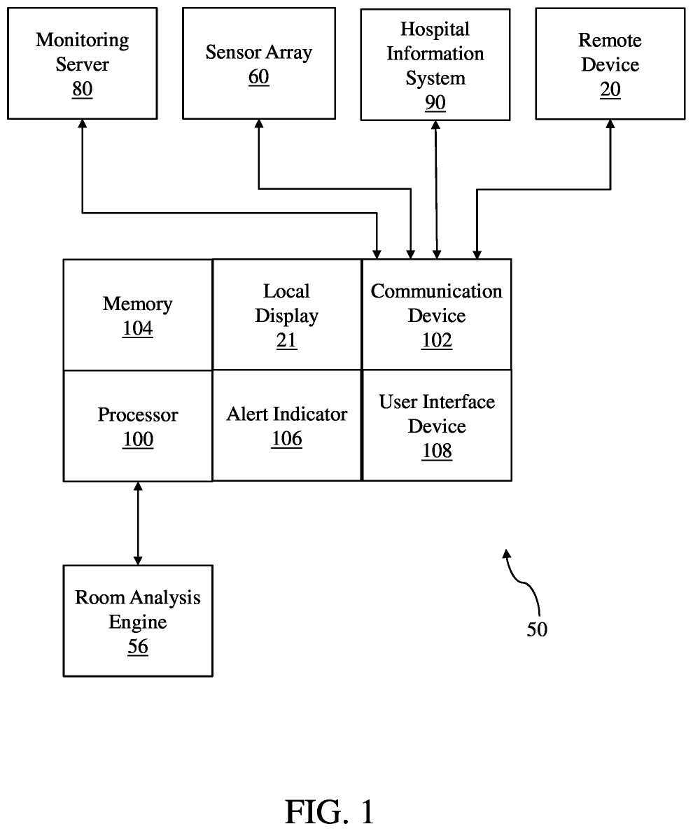

Turning now to the figures, FIG. 1 shows an exemplary operating room monitoring system (ORMS) (50). The ORMS (50) may be implemented as one or more computers, proprietary computing devices, or virtual computing environments. The ORMS (50) includes a processor (100) and a memory (104) that are each located locally and/or remotely to the ORMS (50). Processor (100) in some embodiments is a microcontroller or general-purpose microprocessor that reads its program from memory (104). Processor (100) may be comprised of one or more components configured as a single unit. Alternatively, when of a multi-component form, the processor may have one or more components located remotely relative to the others. One or more components of the processor may be of the electronic variety including digital circuitry, analog circuitry, or both. In some embodiments, the processor is of a conventional, integrated circuit microprocessor arrangement, such as one or more CORE i5, i7, or i9 processors from INTEL Corporation of 2200 Mission College Boulevard, Santa Clara, Calif. 95052, USA, or BEEMA, EPYC, or RYZEN processors from Advanced Micro Devices, 2485 Augustine Drive, Santa Clara, Calif. 95054, USA. In alternative embodiments, one or more reduced instruction set computer (RISC) processors, application-specific integrated circuits (ASICs), general-purpose microprocessors, programmable logic arrays, or other devices may be used alone or in combinations as will occur to those skilled in the art.

Likewise, memory (104) in various embodiments includes one or more types such as solid-state electronic memory, magnetic memory, or optical memory, just to name a few. By way of non-limiting example, memory (104) can include solid-state electronic Random Access Memory (RAM), Sequentially Accessible Memory (SAM) (such as the First-In, First-Out (FIFO) variety or the Last-In First-Out (LIFO) variety), Programmable Read-Only Memory (PROM), Electrically Programmable Read-Only Memory (EPROM), or Electrically Erasable Programmable Read-Only Memory (EEPROM); an optical disc memory (such as a recordable, rewritable, or read-only DVD or CD-ROM); a magnetically encoded hard drive, floppy disk, tape, or cartridge medium; a solid-state or hybrid drive; or a plurality and/or combination of these memory types. Also, the memory in various embodiments is volatile, nonvolatile, or a hybrid combination of volatile and nonvolatile varieties.

Computer programs implementing the methods described herein will commonly be stored and/or distributed either on a physical distribution medium, such as CD-ROM, or via a network distribution medium such as an internet protocol or token ring network, using other media, or through some combination of such distribution media. From there, they will often be copied to a hard disk, non-volatile memory, or a similar intermediate storage medium. When the programs are to be run, they are loaded either from their distribution medium or their intermediate storage medium into the execution memory of the computer, configuring the computer to act in accordance with the method described herein. All of these operations are well known to those skilled in the art of computer systems.

A local display (21) may be proximate to the ORMS (50) and operable by the processor (100) to display interfaces and information to users of the ORMS (50). A user interface device (108) may be proximate to the ORMS (50) and operable by a user to provide user inputs to the ORMS (50). The user interface device (108) may be, for example, a mouse, keyboard, touchscreen display (e.g., the local display (21)), voice activation, or one or more other interface devices. User-facing portions of the ORMS (50) that may be present within an operating room (e.g., the local display (21), the user interface device (108)) may be adapted for use in sterile environments, which may include sterile coverings or coatings, specialized interface devices that are usable with gloves or voice, or other features. An alert indicator (106) may be one or more of a visual display (e.g., the local display (21)), a light indicator, a sound indicator, or another device operable to provide a human-perceptible notification that can alert personnel to the detected circumstances.

A communication device (102) may include one or more components such as WI-FI transceivers, BLUETOOTH transceivers, ethernet adapters, USB adapters, and other wireless and wired connections capable of transmitting data and/or power. The communication device (102) may place the processor (100) of the ORMS (50) in communication with additional devices and data sources, which may include a sensor array (60), a monitoring server (80), a hospital information system (HIS) (90), or a remote display (20). The sensor array (60) may include one or more sensors of varying types that may be distributed in and around an operating room that the ORMS (50) is configured for. The sensor array (60) detects one or more conditions, events, and information about the operating room and may include sensors such as temperature sensors, relative humidity sensors, CO₂ sensors, proximity sensors, motion sensors, vibration sensors, image capture devices, sound capture devices, door position sensors, differential pressure sensors, air flow/velocity and air quality and toxicity sensors, near real-time biological aerosol pathogenic organism detectors, patient physiological sensors, and other types of sensors, depending upon a particular implementation. In various embodiments, one or more of these sensors capture video, and in other embodiments one or more of these sensors will not capture video. Data produced by the sensor array (60) may be received and processed in order to determine various characteristics associated with an operating room, personnel, or patients, for example, as will be described in more detail below.

The monitoring server (80) may include one or more servers (e.g., local physical servers, virtual servers, cloud servers, or other computing environments) that are configured to provide the processor (100) with data and other computing resources to assist in the analysis of data produced by the sensor array (60); to receive, store, and directly process sensor data; or both. As an example, sensor data may include images and/or video captured from within the operating room that is analyzed using machine vision to identify certain objects, people, or other characteristics (e.g., whether a surgeon is wearing a mask and gloves). Analysis for machine vision may be performed by the processor (100), the monitoring server (80), or both. Where machine vision includes aspects of machine learning, the monitoring server (80) may store, update, and maintain data related to the training of recognition, processing, and interface features of the ORMS (50), and the monitoring server (80) may distribute such data and the output of such processing to a plurality of ORMSs (50) so that analysis may be performed locally to each ORMS. In some implementations, the processor (100), the monitoring server (80), or both may be configured to execute and provide a room analysis engine (56) that is configured to receive sensor data and perform specialized analysis thereon (e.g., specialized video and image processing, specialized numerical data processing), as will be described in more detail below. As has been described, varying implementations of the room analysis engine (56) may include features such as machine learning, machine vision, statically configured rule evaluation, globally shared datasets (e.g., machine vision datasets and training datasets), and other features.

The HIS (90) may include one or more servers that are configured to provide the processor (100) and/or the monitoring server (80) with hospital records, patient records, personnel records, and other information usable during analysis of sensor data to monitor for, detect, qualify, quantify, and analyze infection risks. Information provided by the HIS (90) may include, for example, HVAC maintenance records for an operating room, personnel records indicating training, certification, or adherence to operating room guidelines, historic infection rates associated with certain types of procedures or procedures performed in certain operating rooms, scheduling information (such as which patients are to be operated on in which operating rooms at what times), patients' electronic medical records, and other information.

The remote device (20) may include one or more display devices (e.g., LED display) or users' devices (e.g., computers, tablets, mobile devices that include a display and that are configured to interact with the ORMS (50) via a web browser interface, software application, or other interface) located within and remotely from an operating room where the ORMS (50) is configured. As an example, the remote device (20) may include a flat panel display mounted near an operating table or on a wall within the operating room that is configured to display the same information as the local display (21) or a different set of information, as will be described in more detail below. As another example, the remote device (20) may include a web browser interface or software application interface in possession of a hospital administrator and configured to provide high-level information and alerts associated with one or more ORMSs (50). As another example, each person involved in a procedure may possess a remote device (20) (e.g., a smartphone, head-mounted display, wearable device, or proprietary device) so that alerts and information can be individualized to particular users and/or devices. Implementations of the ORMS (50) may have more or fewer components than those shown in FIG. 1, with such other variations being apparent to those of ordinary skill in the art in light of this disclosure.

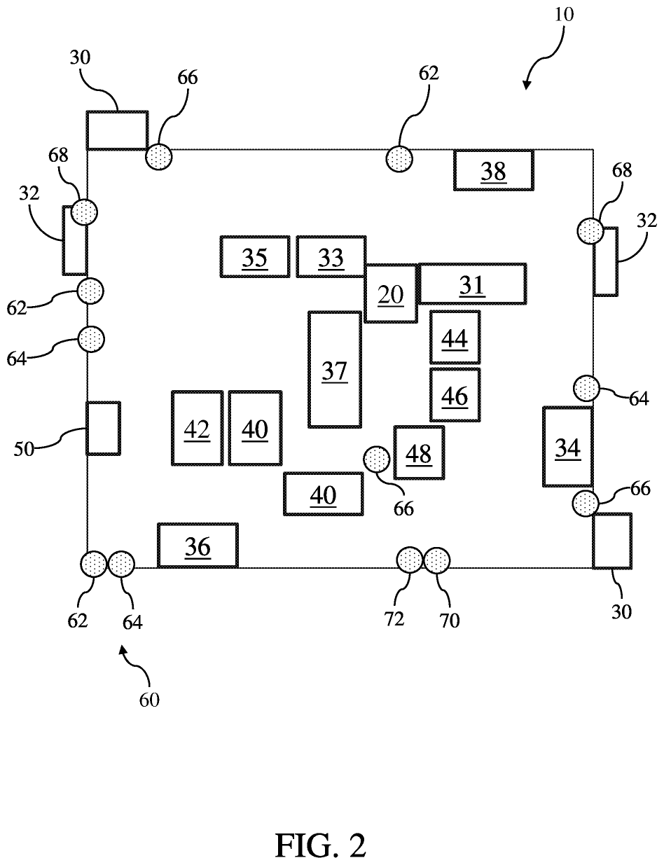

FIG. 2 shows an operating room (10) configured to include an ORMS such as the ORMS (50). The sensor array (60) includes a plurality of sensors positioned around the operating room (10) and configured to detect aberrations in the operating room environment in near real-time. Based on such data, the ORMS (50) may perform various analysis to identify infection risks and provide indications of such risks via the local display (21), the remote device (20), or both. An aberration may include any deviation from optimal room utilization. As an example, an aberration may include positioning one or more back tables (40) outside aseptic designated areas within the operating room (10). In such an example, the present positions of the back tables (40) may be determined based upon locator sensor data such as proximity detection data, wireless triangulation data (e.g., using RFID, BLUETOOTH, or other beacons), optical identification of QR codes or other identifiers, for example. When aberrations are detected, the ORMS (50) may automatically perform tasks intended to address the risk, which may include signaling to the health care providers or surgical team members within the operating room to alleviate the issue detected or, where the aberration relates to an automated device such as a mobile arm, automatically repositioning the mobile arm to mitigate the risk.

While the sensor array (60) of ORMS (50) is distributed around the operating room (10), it should be understood that some implementations of the sensor array (60) may be integrated into a single unit such as a mobile cart or equipment cabinet. As shown in FIG. 2, the sensor array (60) includes a set of cameras (62), a set of proximity sensors (64), a set of air velocity sensors (66), a set of door position sensors (68), a temperature sensor (70) and a relative humidity sensor (72). However, it will be appreciated that in various implementations of the ORMS (50) the number and type of sensors may vary, and certain sensors may be omitted while other alternative sensors may be provided (e.g., a particle counter, a microbial detector) in order to suit particular applications of the technology or implementation environments.

A set of one or more cameras (62) is positioned around the operating room (10) to provide various views despite the likelihood of obstruction by equipment or personnel at times during a procedure. As such, the set of cameras (62) may be mounted near walls, on the ceiling away from a wall, or on equipment within the room (e.g., on the mobile arm (31)).

The set of proximity sensors (64) are positioned around the operating room (10) (e.g., proximate to walls, away from walls on the ceiling, on equipment) to monitor areas associated with infection risks. Data produced by proximity sensors (64) may be used in varying ways. As an example, data from a proximity sensor (64) positioned near a handwashing sink (often in an adjacent room, not shown) may be used to verify the number and/or identity of personnel who spent at least a minimum amount of time near the sink, which may indicate whether the personnel involved in a procedure did or did not wash their hands. As another example, a proximity sensor (64) positioned near anesthesiology equipment may detect that a person (e.g., the anesthesiologist) has moved from their area and may risk spreading contamination into other areas.

The set of air velocity sensors (66) are positioned around the operating room (10) (e.g., proximate to walls, away from walls on the ceiling, on equipment, near doors, near HVAC equipment such as vents (30), and at varying heights along the walls) to monitor the flow of air within the operating room (10). Data produced by the air velocity sensors may be used to determine the speed and direction that air is flowing within the room, which may be useful in determining whether current airflow is desirable (e.g., fresh air is being supplied while airborne contaminants are being exhausted) or undesirable (e.g., normal airflow is being influenced by an opened door, misplaced equipment, or other factors).

The set of door position sensors (68) are placed on doors (32) in order to determine whether the equipped door is open or closed. Open doors may influence infection risks within the operating room (e.g., by disturbing normal airflow and potentially introducing contaminants). Data produced by the door position sensors (68) may be used to determine how many times a door is opened, the length of time that the door remains open, and whether the door is completely shut (e.g., firmly seated within the frame with a mechanical or magnetic latch or other mechanism engaged). Door position sensors (68) may also include other features such as an automatic door opener, automatic door closer, automatic locking mechanism, and door status indicator (e.g., a light indicator or audio indicator) to provide information related to the door. Such features may be used by the ORMS (50) to enforce guidelines and minimize the extent and frequency of door use, automatically close doors that are partially open, or otherwise gather information related to the doors (32).

The temperature sensor (70) and relative humidity sensor (72) may be positioned around the operating room (10) at locations that generally represent the climate within the room or that have particular climate concerns. As has been described, other air and climate quality sensors may also be included in the ORMS (50) as may be desired, such as particle counters, microbial detectors, and other devices. Information produced by such devices may be used to detect undesirable climate and air quality traits, automatically operate devices (e.g., HVAC, air filtration systems) to improve air quality or climate, or to take other action.

The operating room (10) also includes various additional equipment related to the function of the room itself rather than the function of the ORMS (50). Such additional equipment will vary, but may include an anesthesia cart (33), a work station (34), an equipment stack (35), a mobile case cart (36), a surgical table (37), an anesthesia prep cart (38), a set of back tables (40), a set of spec carts (42), a linen cart (44), a perfusion cart (46), and a set of may stands (48), for example. A user-facing portion of the ORMS (50) may also be present in the operating room (10), which may include the local display (21) and the user interface device (108) as well as other components. One or more remote devices (20) may also be present as fixed displays (e.g., such as a display positioned near the surgical table (37) as shown in FIG. 2), carried or worn devices, or other devices, as has been described.

Based on the variety of equipment and component that may be present in any individual room, it can be seen that it may be advantageous to provide a flexible monitoring system that may be adapted to a particular room. This may also be true for procedures and guidelines as each unique operating room, including operating room (10), may be associated with a predetermined infection-reduction protocol, such as by using the methodology described in Methodology for Analyzing Environmental Quality Indicators in a Dynamic Operating Room Environment, Am. J. of Infection Control; 45 (2017) 354-9, the entirety of which is hereby incorporated by reference.

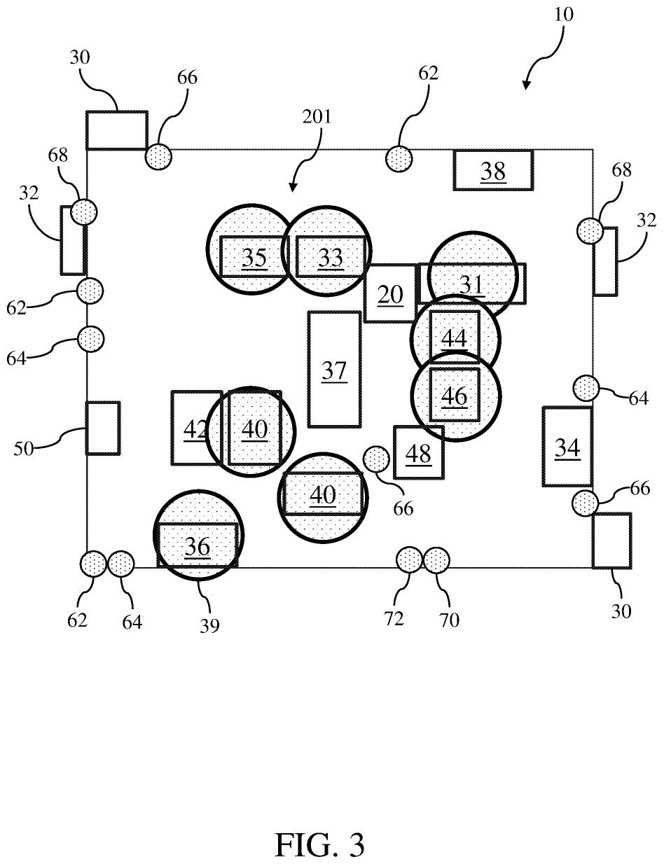

Monitoring and enforcement of guidelines may include configuring the ORMS (50) to define optimal positions for various equipment in the room. With reference to FIG. 3, a set of patterned circles are shown overlaid upon the operating room (10) to indicate optimal areas within which certain equipment should be placed. For example, the mobile case cart (36) can be seen as substantially contained by an optimal zone (39), which is represented as a patterned circle. Defined areas may be configured manually for the ORMS (50) when the operating room (10) is initially configured, or they may be automatically configured based upon known characteristics of the room (e.g., a single value or pair/set of coordinates indicating the position and orientation of the surgical table (37) within the operating room (10) may provide an anchor for relative positions where each other piece of equipment should be ideally placed). While FIG. 3 illustrates a configuration associated with the ORMS (50) and the operating room (10), it may also be displayed as a user interface via the local display (21), a remote device (20), or both. Such an interface may aid in positioning equipment prior to a procedure, positioning equipment during a procedure in response to an alert from the ORMS (50), and in other tasks. For example, where a wireless triangulation or other position determination is made for the mobile case cart (36), its current position may be displayed within the operating room (10) relative to the optimal zone (39).

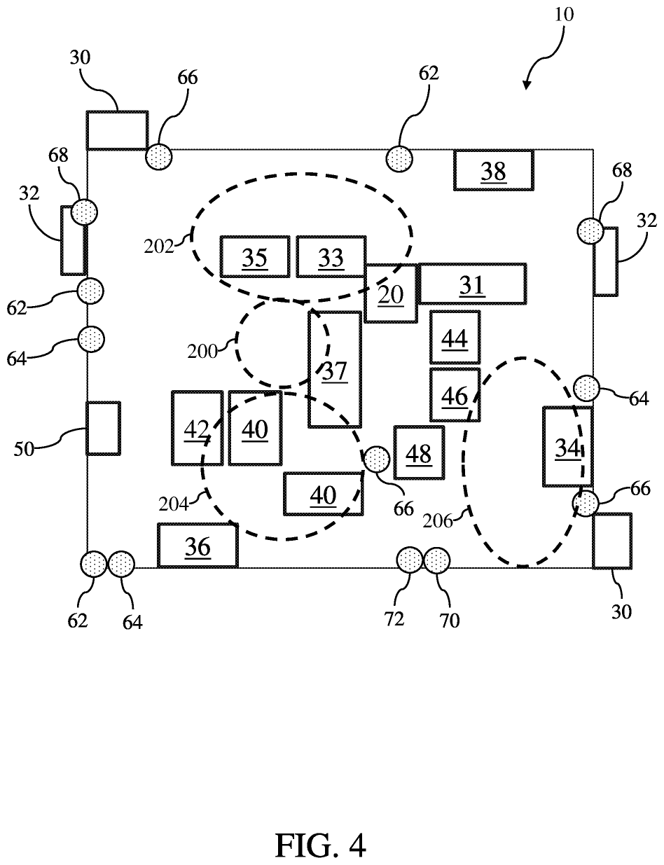

The ORMS (50) may also be configured to define optimal positions or areas where certain personnel should move within the operating room (10). With reference to FIG. 4, several such areas are overlaid upon the diagram of the operating room (10) as dotted circles or ovals. A surgeon area (200) is positioned proximately to the surgical table (37) and defines an area in which a surgeon should ideally remain in order to reduce the spread of bacteria and limit the chance of infection. An anesthetist area (202) is located proximately to the anesthesia cart (33) and defines a similar area for an anesthetist. A nurse tech area (204) is positioned proximately to the back tables (40) and defines a similar area for a nurse tech assisting in a procedure. A circulating nurse area (206) is positioned proximately to the workstation (34) and defines a similar area for a circulating nurse assisting in a procedure. As with optimal equipment locations, the personnel-specific areas may be manually configured or may be determined automatically based on a position of the surgical table (37) or other equipment. Personnel areas may be used to determine the spread of bacteria and calculate resulting infection risk, to provide alerts or other indications when personnel have left a designated area (e.g., based on an alert from a proximity sensor (64)), or to take other actions. As with prior examples, an interface similar to FIG. 4 may be displayed to aid personnel in learning or maintaining an optimal position and spacing within the operating room (10), to alert personnel when they exit their ideal space and aid them in returning, or to provide other information.

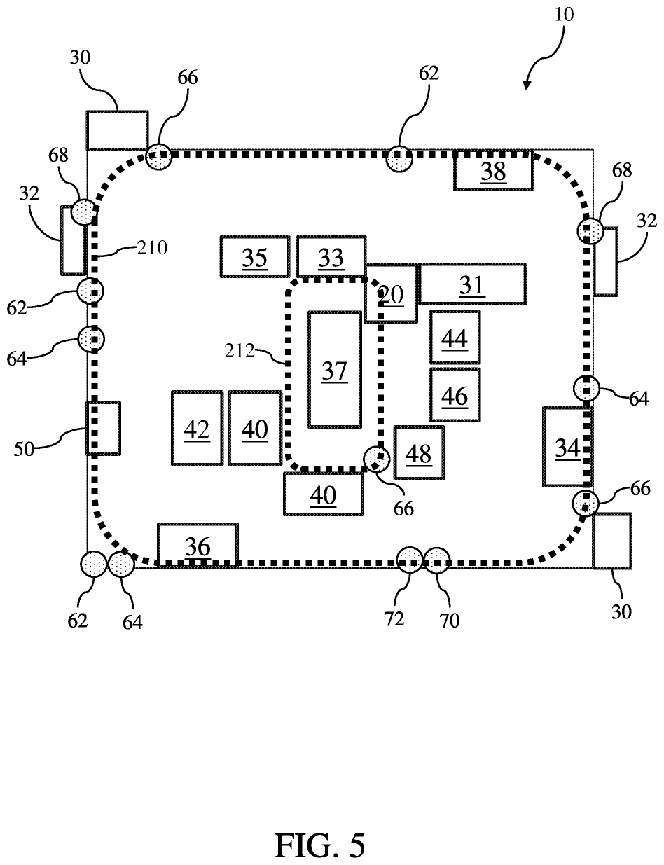

The ORMS (50) may also be configured to define different air quality zones within the operating room (50), with such configurations being manually performed or automatically performed based upon the position of particular equipment, such as the surgical table (37), HVAC equipment and vents (30), low wall-mounted exhaust vents and filtration systems (not pictured), or other equipment. With reference to FIG. 5, the operating room (10) is shown with different air quality zones overlaid thereon as dotted boxes. A contaminated air zone (210) is shown as including much of the operating room (10), indicating a space where, due to personnel, equipment, airflow, or other factors, there is an elevated chance of airborne contaminants being present. A clean air zone (212) is shown within the contaminated air zone (210), indicating an area within the operating room (10) where airflow and other factors result in a lowered chance of airborne contaminants being present. Such configurations may be used to determine the impact on the risk of infection that aberrations or other deviances in procedures and guidelines may have.

For example, where a surgeon passes a surgical instrument from the clean air zone (212) to the contaminated air zone (210), the ORMS (50) may detect an aberration. However, there may be no substantial change to risk of infection unless the instrument were to re-enter the clean air zone (212) without being re-sterilized. As another example, where the surgeon completely leaves the clean air zone (212) and then returns without first rewashing and re-sterilizing (e.g., as may be detected by a proximity sensor (64) near a sink), there may be a substantial increase in the calculated risk of infection. As with prior examples, an interface may be displayed showing information such as that shown in FIG. 5. Such an interface may be dynamically updated to show positions of equipment and personnel as they work in the operating room, and to change the size, shape, and/or position of the clean air zone (212) as a result of potentially contaminated personnel or equipment passing therein, as a result of a failure or malfunction in the HVAC or other exhaust and filtration systems, or in other cases.

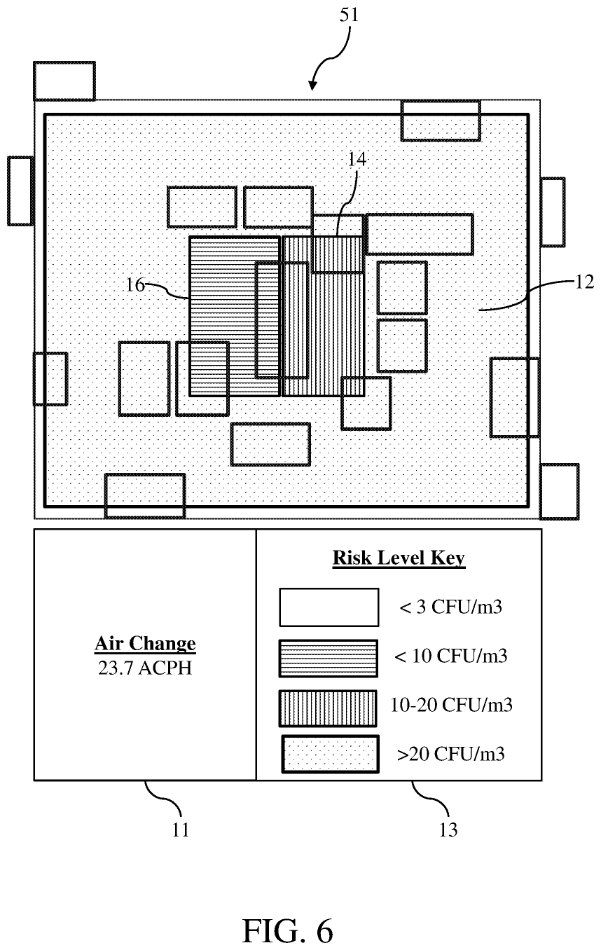

One or more configurations, such as those described above and others, may be used to define an infection-reduction protocol for each unique operating room such as the operating room (10). As an example, FIG. 6 shows a screenshot of an exemplary interface that may be displayed via the local display (21) or any remote device (20) to provide an indication of zones for which the infection-reduction protocol provides certain requirements. The interface includes a contamination risk picture (51) overlaid upon the operating room (10). The interface also includes an air change indicator (11), which provides information relating to the flow, filtration, and changing of air within the operating room (10), and a risk level key (13) which provides information usable to visually distinguish and interpret zones in contamination risk picture (51). The contamination risk picture (51) depicted in FIG. 6 shows a large portion of the operating room (10) designated as a high-risk area (12), a smaller area right-of-center designated as a medium-risk area (14), and a smaller area left-of-center designated as a low-risk area (16).

The high-risk area (12) may be an area where air quality is low compared to the rest of the operating room (10), as may be determined based upon configured information such as that shown in FIG. 5. As such, the infection-reduction protocol may require that any surgical instrument or surgical device present within this area must be covered or sealed within a sterile space or must be sterilized prior to leaving the high-risk area (12). The protocol may similarly require that any personnel within the high-risk area (12) scrub and re-sterilize before leaving the high-risk area (12). A multitude of other requirements may be predetermined and associated with the high-risk area (12) in order to provide a set of rules and criteria that data gathered from the sensor array (60) may be evaluated against, as will occur to those skilled in the art.

The medium-risk area (14) may be associated with a different set of procedures, rules, and requirements in a second infection-reduction protocol for medium-risk areas. Such requirements may allow hospital staff and covered instruments to be freely placed within or to pass through the area without violating the second infection-reduction protocol. However, such instruments and personnel passing into the high-risk area (12) may violate procedures associated with the infection-reduction protocol for that area.

The low-risk zone (16) may be associated with a still different set of procedures, rules, and requirements in a third infection-reduction protocol for low-risk areas. Such areas may have the most permissive requirements, and thus may be defined within the third infection-reduction protocol as the only location(s) where the surgical table (37), the back instrument tables (40), the Mayo stands (48), and uncovered instruments and/or implants and other critical equipment may be placed. As can be seen, defining and configuring the ORMS (50) with an infection-reduction protocol (e.g., one including a different sub-protocol specific to each risk area type) provides a set of rules that may be evaluated and applied to each area based on data received from the sensor array (60). Thus, while the placement of sensors and the configuration of a particular operating room (10) as illustrated in FIGS. 2-5 may vary depending on each unique room, the infection-reduction protocol may stay largely constant between rooms and may be applied based upon the static or dynamic determination of areas within a particular room.

With that context, it can be seen that the risk level key (13) provides an indication of the number of bacteria (e.g., in terms of colony forming units, or CFU) in each cubic meter of space within the operating room (10). Risk level may be determined across the room for each different zone, and the contamination risk picture may be updated accordingly based upon data from the sensor array (60). This also provides a scale by which aberrations or deviations from an infection-reduction protocol may be analyzed to determine their impact on risk level. For example, the ORMS (50) may be configured to associate CFU values with different equipment, surgical instrument, or personnel. When equipment with a configured CFU value is introduced into a risk area of higher CFU value, there may be no change in the risk picture (51). However, when the equipment is introduced into area with a lower CFU value, the risk picture (51) may be recalculated and redisplayed to reflect the potential introduction of CFU into the lower risk area. Such determinations may be performed as estimates, may rely upon the sensor array (60) (e.g., a microbial detector), or both, as will be apparent to those of ordinary skill in the art in light of this disclosure.

The infection-reduction protocol may also include other rules which are more general or global in their application. For example, the infection-reduction protocol may be configured to mimic a set of one or more operating room guidelines regardless of risk area, such as: all hospital staff must scrub in upon entering the operating room; all hospital staff must wear proper gowns during the operation; all hospital staff must wear a facemask and maintain it in the proper position; all hospital staff must wear shoe covers; all hospital staff must wear hair coverings; doors to the operating room must remain closed; the temperature must be maintained within a specified range; the relative humidity must be maintained within a specified range; the airflow rate (at one or more locations) must be maintained within a specified range; the number of occupants in the operating room must fall between a minimum number and a maximum number; diathermy or electrocautery devices must be used with a vacuum; and other guidelines, as will be apparent to those of ordinary skill in the art in light of this disclosure. As has been described, sensor data collected from the sensor array (60) may be evaluated in view of each such global guidelines of the infection-reduction protocol in order to identify aberrations, update the risk picture (51), provide alerts, and take other actions.

With reference to FIG. 1, it can be seen that the individual sensors of the sensor array (60) of ORMS (50) may be positioned at locations that enable the ORMS (50) to gather data to be evaluated against one or more of the rules defined within the global or area-specific infection-reduction protocol. In this manner, the infection-reduction protocol may guide the configuration of the ORMS (50) within the operating room (10). In some implementations, the ORMS (50) may be configured to provide an interface to aid in initial configuration by receiving inputs from a user defining static characteristics of the operating room (10) (e.g., defining wall positions, ceiling height, door positions, HVAC vent positions, etc.). Based upon such inputs, and for each unique operating room (10) and infection-reduction protocol, the ORMS (50) could produce a diagram indicating the positions at which each individual sensor of the sensor array (60) should be placed to enable data collection.

Using the exemplary global guidelines provided above, it can be seen that the placement of sensors is advantageously related to the infection-reduction protocol. For example, the camera(s) (62) are positioned around operating room (10) so as to provide a partial or complete view of the room from a number of different angles so as to prevent any obstruction in views which may occur from movement of hospital staff and/or equipment. In some implementations, cameras (62) may be mounted to the ceiling or other equipment to provide, in the aggregate, substantially unobstructed fields of view. In some implementations, the set of cameras (62) provide for a viewing angle equal to or greater than 60 degrees. In some implementations, some cameras in the set of cameras (62) may be mounted centrally within the operating room (10) and provide a viewing angle equal to or greater than 90 degrees. In some implementations, the set of cameras (62) may be combined as an array to provide for a 180-degree or 360-degree field of view. The set of cameras may provide high-definition video surveillance to the ORMS (50), or may provide another resolution, framerate, or other video or image characteristic as may be desired for a particular application. As has been described, captured images, video, audio, and other data may be provided to the processor (100) via a wired or wireless connection with the communication device (102) and may pass through one or more intermediary devices, such as a video processor (e.g., to modify video quality or apply automated anonymization) or storage device, if desired.

The set of proximity sensors (64) as shown are located near high-risk areas within the operating room (10) as well as near the doors (32) and scrub-in station. These sensors provide data that may be used in assessing compliance with one or more of the rules outlined in the infection-reduction protocol, such as tracking movement of equipment and personnel between zones and monitoring compliance with washing procedures and other requirements. For example, the proximity sensors (64) may produce data usable to ensure compliance with other rules, such as scrub-in requirements upon entering the operating room and/or the maximum number of hospital staff permitted within the operating room. In some implementations, the proximity sensors (64) each emit an electromagnetic field or a beam of electromagnetic radiation (infrared, for instance), and monitor the different elements for changes in the field or return signal. In some implementations, one or more capacitive proximity sensor(s) or photoelectric sensor(s) may be used.

The air velocity sensors (66) may produce data usable to ensure that the proper number of ACH occur in order to comply with the minimum guidelines required by medical standards and/or that specified in the infection prevention protocol. In some implementations, the air velocity sensors (66) are air flow transducers which provide for enhanced sterility when compared to conventional fan type airflow sensors as they do not require larger surface areas, such as blades, which allow for dust and other contaminants to accumulate. The air velocity sensors may be placed near doors (32), near vents (30), and in other areas where air flow may be of particular interest in applying the infection-reduction protocol.

The sensor array (60) also includes a door position sensor (68) for each door (32), a temperature sensor (70), and a relative humidity sensor(s) (72). The door position sensors (68) are operable to detect the opening and closing of an equipped door (32). Door position sensors (68) may be one of a variety of known types, including magnetic sensors, mechanical switches, or the like. Temperature sensors (70) preferably measure the air temperature within the zone of the operating room in which they are placed. These temperature sensors (70) may be, for example, negative temperature coefficient thermistors, resistance temperature detectors, thermocouples, semiconductor-based sensors, or any other type of temperature sensor suitable for use in an operating room. The humidity sensors (72) measure the relative humidity in the zone of operating room (10) in which they are located. These humidity sensors (72) may be designed so as to utilize capacitance, resistance, thermal conductivity, or other characteristics in order to measure the relative humidity.

As has been described, contextual or object proximity sensors, radio-frequency identification (RFID), BLUETOOTH, and other wireless triangulation devices can be utilized to confirm that surgical tools or carts are placed in the most antiseptic or optimal position, as specified by the infection-reduction protocol (e.g., as shown in FIG. 3). Such data may be used by the ORMS (50) to determine their current positioning and overlay those positions on the room contamination risk picture (51) to inform in-room users of real-time risks. As such, the ORMS (50) may issue an alert to correct the positioning of a positionally tracked device or equipment when it is outside of an ideal position, according to an infection-reduction protocol.

As with captured image data, the signals resulting from any of the other set of sensors (60) may be provided to the processor (100) via a wired or wireless connection to the communication device (102). In some implementations, one or more of the in-room context or environmental sensors within the set of sensors (60) may be integrated with one another as placeable sensor modules. In some implementations, the ORMS (50) is a self-contained unit which includes a number of built-in and remote but wireless sensors that may be placed. The wireless sensors may operate using one of a number of known wireless standards, such as BLUETOOTH or IEEE 802.11.

As has been described, the ORMS (50) evaluates data from the sensor array (60) with one or more rules provided by the infection-reduction protocol. For example, ORMS (50) may trigger an alert if door position sensor (68) signals that the door is opened for greater than a specified number of seconds (e.g., 10 seconds). In some implementations, an alert may be issued if the door is opened more than a specified number of times during a given operation. As another example, humidity sensor (72) may periodically or continuously monitor the relative humidity in the operating room (10). In the event the humidity falls below or rises above a level or range provided in the infection-reduction protocol, an alert may be issued, or other corrective action may be automatically implemented.

In other cases, one or more of the sensors within sensor array (60) may work in conjunction with live video analysis in order to determine compliance with a rule in the infection-reduction protocol. For example, upon entry into a room by a hospital staff member, data from a door position sensor (68) may initially indicate to the ORMS (50) that someone has entered or left the operating room. The video information provided by the camera(s) (62) may subsequently be analyzed to confirm what actually occurred and to determine what, if any, impact the event may have on the infection-reduction protocol.

As has been described, the ORMS (50) may include specialized machine vision configurations (e.g., the room analysis engine (56) and/or data from the monitoring server (80)) to aid in object recognition, which may include an event identifier for identifying particular events that occur in association with the identified objects, an artificially intelligent engine (such as a machine learning engine that is trained upon suitable video segments) for determining whether each particular event or combination of events affects compliance with an infection-reduction protocol developed for the hospital operating room. ORMS (50) may also include an alarm indicator for issuing an alert (e.g., via the alert indicator (106) or remote devices (20)) when any particular event or combination of events is determined to be non-compliant with said infection-reduction protocol. This method of determining compliance may be used in conjunction with other detection methods using other sensors, or it may operate based on data from a single detection method.

Captured images and video usable in machine vision processes may combine the different views from one or more of the set of cameras (62) so as to generate a single concatenated video information feed, or it may correlate certain areas of each video information feed with one another, such as where multiple cameras cover the same area. Image capture may also include pre-processing of the incoming image data, such as to correct brightness, contrast, or the like. Image processing may also include masking of known background objects, which can reduce the processing needed in subsequent steps and aid in the identification of material objects within the video. Such a process can be performed efficiently where the ORMS (50) has been configured with certain details about a unique operating room (10) since such details largely remain static. Examples of static features that the ORMS may be configured to account for and mask may include walls, lights, doors, switches, cabinets, and other permanent or generally static features of operating room (10).

In some implementations, image processing may also include masking out or otherwise anonymizing facial features or patient anatomy, and may also mask out confidential information (e.g., such as patient information displayed on a video monitor). As with prior video processing examples, known locations of video monitors or other information sources may be used as inputs to aid in identifying and masking such data. Similarly, known identities of patients, practitioners, and others may be used as inputs to aid in identifying and masking facial features, identity data, or patient anatomy.

During machine vision analysis, the processor (100), the monitoring server (80), or both (e.g., using the room analysis engine) can then subsequently process the image information using an object recognizer to recognize foreground objects present in the video information. In some implementations, the machine vision engine utilizes a predictive modeling engine, such as indoor air quality (IAQ) analytics engines like those described in Jagriti Saini, et al. (Saini, J., Dutta, M., & Marques, G. (2020). A Comprehensive Review on Indoor Air Quality Monitoring Systems for Enhanced Public Health, Sustainable Environment Research, 30(6), https://doi.org/10.1186/s42834-020-0047-y), incorporated herein by reference, which measured 2.5 micrometer-size particles to control airflow for optimum public health in buildings. Objects identified may include a surgical table, a surgeon, an equipment stack, an anesthesia cart, an anesthesiologist, a back table, a mobile base cart, a Mayo stand, a spec cart, a nurse, a workstation, any subcomponent thereof, any surgical tool, implant, or the like lying thereon, or any other medical equipment.

During machine vision analysis, the room analysis engine (56) may include a machine learning function that focuses on characteristics or events associated with identified objects, while a machine vision analysis focuses on the identification of various foreground objects. For example, the machine learning process may be trained on images of a surgeon in properly and improperly worn surgical gowns, including headwear and a mask, to improve subsequent recognition of proper and improper technique. Still further, the machine learning process may be trained on images of a human having a mask properly and improperly positioned over their mouth and nose, thereby enabling the machine learning process to detect the improper lowering of a face mask during an operation, or the entry into the room of a hospital staff member who is either not wearing a face mask or is wearing the face mask in an improper position (e.g., around the neck).

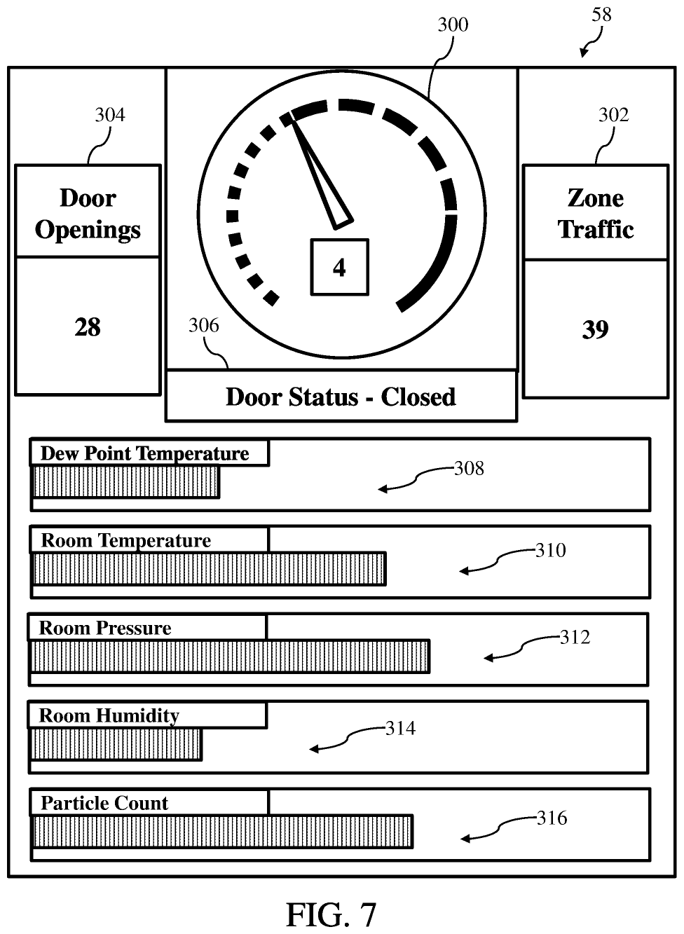

When providing real-time information and alerts related to monitoring (e.g., an indication that a door is opened, an alert that personnel are not wearing masks), the ORMS (50) may display various interfaces to provide instruction, information, and other functionality to personnel that may be used to address the aberration or mitigate the resulting risk of infection. As an example of such an interface, FIG. 7 shows an environmental quality index (EQI) dashboard (58). The EQI dashboard (58) may be displayed via the local display (21), the remote devices (20), or both. The EQI dashboard includes a quality gauge (300) configured to provide, based upon data from the sensor array (60), information relating to air quality within one or more portions of the operating room (10). The quality gauge (300) may utilize color gradients, patterns, or other visual distinctions to communicate EQI data, and it may also display such information in numerical forms. A traffic indicator (302) provides an indication of a number of instances that sensors have detected traffic or movement in one or more critical areas (e.g., near sinks, near sources of contamination, near transition borders between risk areas). One or more traffic indicators may be displayed via the dashboard (58) as may be desired. For example, some implementations of the dashboard (58) may show a number of detected persons that have entered the operating room (10) and the number of persons detected near the sink, such that a difference may indicate that one or more personnel have not followed washing procedures.

A door indicator (304) may be configured to display and provide an indication of a number of times that doors have been opened, an average length of time that they have remained open, and other information. A door status (306) may be configured to display and provide an indication of whether the door is currently open. Other indicators beyond the traffic indicator (302) and the door indicator (304) may be provided or configured by a user as may be desired to present information via the dashboard (58), as will be apparent to those of ordinary skill in the art in light of this disclosure.

A number of additional gauges are also shown as part of the dashboard, including a dew point temperature gauge (308), a room temperature gauge (310), a room pressure gauge (312), a room humidity gauge (314), and a particle count gauge (316). As with other examples, such gauges may be colored, patterned, or otherwise visually distinguished in their display to indicate their criticality as it relates to infection risk.

In some implementations, the dashboard (58) may be dynamically displayed to a plurality of recipients based upon their identity, based upon their unique configurations, or based upon other factors. For example, each of a circulating nurse and a nurse tech may possess a remote device (20) that emits or responds with a signal (e.g., a visual pattern, electromagnetic pattern, or RFID beacon identifier) that is uniquely associated with them. The dashboard (58) may automatically provide varying information sets to each remote device (20) based upon a global configuration, or it may provide customized information to each remote device (20) based upon manual configurations provided by users of the device.

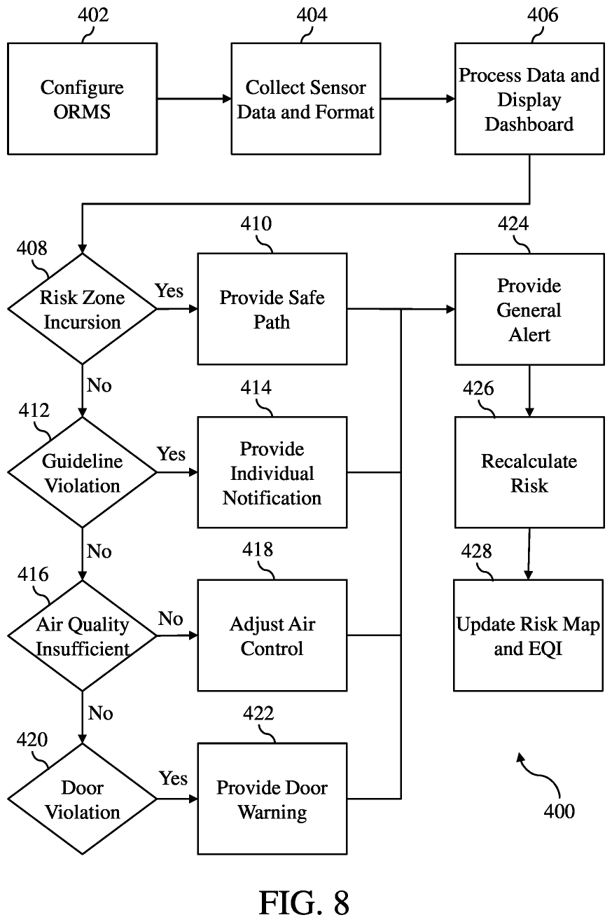

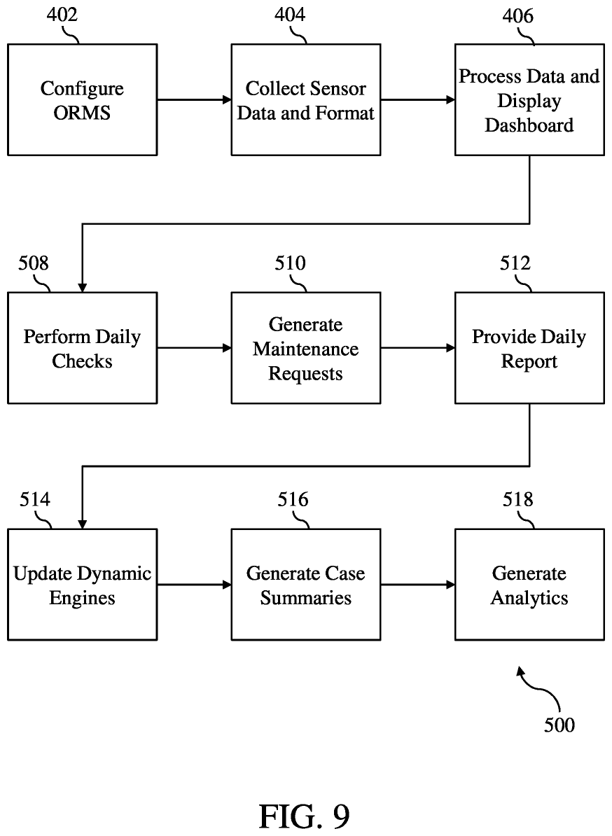

While various features and functions of the ORMS (50) have been described, FIGS. 8 and 9 provide more detailed flow charts illustrating several use cases possible with the ORMS (50). FIG. 8 shows a flowchart of an exemplary set of steps (400) that may be performed with an operating room monitoring system such as the ORMS (50). After the ORMS (50) is configured (402) for a particular operating room, which may include placing and configuring the sensor array (60), setting up the local display (21) and any remote devices (20), communicating with the monitoring server (80) and HIS (90), and receiving an infection-reduction protocol, sensor data may be collected (404) from the sensor array (60) and formatted for subsequent use (e.g., image background masking, image anonymization, conversion of sensor data into structured formats). Received data may then be processed (406) and analyzed using the infection-reduction protocol in order to identify any deviations from the protocol, and then sensor data and analysis results may be displayed via an interface such as one or more of those shown in FIGS. 3-7. In some embodiments, processing may also include correlating data from the sensor array (60), metadata regarding that data, EQI data streams, and the like with subsequent infection data and using the correlation to further train ORMS (50).

As has been previously described, the ORMS (50) may perform additional actions based upon whether the processing and analysis (406) of data from the sensor array (60) identifies any deviations from the infection-reduction protocol. As an example, a general category of deviation response is based on whether there is a risk zone incursion detected (408) within the operating room (10). This could include a person, piece of equipment, surgical instrument, or other object resting within or moving into an area within the operating room (10) that may increase the risk of infection. As a further example, this could include a person moving outside of a designated area (e.g., a circulating nurse leaving the area (206)), or a piece of equipment moving from a first risk area into a lower risk area (e.g., the perfusion cart (46) moving from the high-risk area (12) into the medium-risk area (14)).

When such a risk zone incursion is detected (408), the ORMS (50) may determine and provide (410) a safe path or other corrective action that may be performed to reduce or mitigate the risk of infection related to the incursion. This may include displaying via one or more local or remote devices (20) a textual description or a representation of the operating room (10); an identification of the person, equipment, or object associated with the risk; and identifying a path to a destination that the person, equipment, or object should follow to reverse or mitigate the effect of the incursion. The safe path may be provided (410) to each remote device (20) or other display or may only be provided to an individual responsible for mitigating the incursion. For example, where a surgeon places an instrument in an elevated risk area, a notification may be provided (410) to a user device associated with a nurse tech that identifies the object and describes where the object should be placed or moved to and how it should be transported. As an additional example, where a nurse tech moves into a lower risk area and causes an incursion (408), a notification may be provided (410) to a remote device (20) associated with that nurse tech.

Another general category of action taken by the ORMS (50) may be triggered by violation of guidelines (412) provided by an infection-reduction protocol, such as a failure to wash hands, failure to wear a mask, placement of an uncovered or unsealed instrument in a contaminated area, excessive personnel within the operating room, or other examples that have been described herein or will be apparent to those of ordinary skill in the art in light of this disclosure. Such violations may be detected in various ways as has been described (e.g., image analysis to identify improper mask placement, image analysis or wireless triangulation to locate instruments, proximity tracking at doorways and wash areas, etc.). When detected (412), the ORMS (50) may provide (414) a notification of the violation to each remote device (20) or to a subset of remote devices (20) that identifies the violation and any actions that should be performed to mitigate the violation.

Another general category of action taken by the ORMS (50) may be triggered by some aspect of air quality or climate within the operating room (10) falling (416) outside of desirable or sufficient ranges. This may include deviations in temperature, humidity, pressure, magnitude and direction of air flow, particle count, microbial count, or other characteristics, as may be detected by the sensor array (60). When detected (416), the ORMS (50) may provide notifications to responsible parties as has been described, or it may automatically adjust (418) or operate HVAC equipment or other systems (e.g., through direct communication or communication via the HIS (90)) to correct the air quality issue. This may include adjusting ACH rates, increasing or decreasing temperature or humidity, activating additional exhaust vents or air filtration systems, or other actions.

Another general category of action taken by the ORMS (50) may be triggered based upon violation (420) of protocol related to doors (e.g., room entry doors, cabinet or equipment doors, or other fixtures that may be equipped with a sensor such as the door position sensor (68)). This may include frequent or prolonged opening of the door (32), failures to completely close the door (32), failures to close doors or other seals on equipment (e.g., such as closing a cabinet door where sterile instruments are stored), and other scenarios related to the infection-reduction protocol that may be detected by the sensor array (60). When detected (420), the ORMS (50) may provide (422) a door warning to one or more remote devices (20) (e.g., such as a device mounted near the door (32)) indicating that the door should not be opened or that opening of the door should be delayed for some period of time (e.g., to allow air quality to be corrected after prior openings). In some implementations this may also include operating a door closer to close the door or performing other automated actions to comply with the infection-reduction protocol.

While various circumstances can be detected as violations and trigger notification and/or other action (e.g., such as incursions (408), guideline violations (412), air quality violations (416), and door violations (420)), the ORMS (50) may also be configured to only take action after detection when the circumstance exceeds a certain threshold or magnitude. Technical violations that do not exceed the threshold may be ignored or may be prioritized for processing of future analysis tasks (e.g., data associated with the technical violation may be received and processed at an increased priority relative to other tasks of the processor (100), the monitoring server (80), or other processors). Such threshold configurations may be defined at the level of the infection-reduction protocol (e.g., one person improperly wearing a mask may not be defined as an aberration, while two or more people improperly wearing masks is an aberration), but may also be configured separately from the infection-reduction protocol (e.g., such as by manual user configuration specific to a particular operating room, procedure, or facility). Such thresholds may additionally be used to determine varying levels of response to aberrations. For example, an occurrence that is technically an aberration but is of low risk may trigger actions related to daily report generation (512), case summaries (516), and analytics (518), but may not generate general alerts (424). A higher-risk aberration may trigger various administrative actions, and may additionally trigger tailored responses (e.g., individual notifications (414)) and general alerts (424) (e.g., visual or auditory feedback from the alert indicator (106)). As with prior examples, such configurations may be defined as part of the infection-reduction protocol or may be manually configured for a particular operating room, procedure, or facility, for example.

In addition to providing various tailored responses to identified aberrations, the ORMS (50) may also provide (424) a generalized alert, which may include displaying the alert on the local display (21) or one or more remote devices (20), operating the alert indicator (106) to signal the occurrence of an aberration, providing notifications to personnel outside of the operating room (10), or other alerts. The ORMS (50) may also recalculate (426) various parameters related to infection risk based upon the detected aberration. This may include updating and recalculating characteristics of the room, such as the risk areas of FIGS. 5 and 6, recalculating EQI, recalculating measured or estimated CFU, and other determinations. As an example, with reference to FIG. 6, where an aberration results in the introduction or potential introduction of contaminants to the medium risk zone (14), the size, shape, and position of the medium risk zone (14) may be changed to indicate the risk of entrainment of bacteria. As another example, the colors, patterns, or other visual characteristics of the risk picture (51) may be updated to reflect an increased CFU count in one or more areas of the operating room (10). As yet another example, the EQI shown in the quality gauge (300) may be updated to reflect a decrease in air quality in one or more areas within the operating room (10). As still another example, contamination risk estimates may be calculated for various areas within the operating room (10).

Once risks have been recalculated (426), one or more interfaces, such as the EQI dashboard (58), may be updated (428) to display updated room characteristics, to display changed levels of risk across risk areas, or to display changes in size and/or location of risk areas, for example. As can be seen, repeating one or more of the described steps provides near-real-time monitoring, notification, and mitigation of aberrations within the operating room.

As another example of features and functions provided by the ORMS (50), FIG. 9 shows a flowchart of an additional exemplary set of steps (500) that may be advantageously performed across a hospital environment having a plurality of ORMS (50). As with the steps of FIG. 8, after one or more ORMS (50) are configured (402), sensor data is collected (404), and data is processed (406), data provided by the plurality of ORMS (50) may be used in various ways.

This may include performing (508) automated daily checks of each operating room by gathering and analyzing sensor data to verify the function of HVAC equipment, verify the presence of required equipment, verify the arrangement of equipment, check sensor operation, and monitor other characteristics. This may also include performing (508) automated daily checks related to personnel, which may be used to provide individualized reports or suggestions based on prior participation in procedures (e.g., a suggestion to review proper mask placement and fitment procedures based on past detected deviations from protocol).

As another example, data collected by the plurality of ORMS (50) may be used to automatically generate (510) maintenance requests. This may include creating electronic tickets or sending electronic communications related to the repair, maintenance, or replacement of medical equipment or HVAC equipment, calibration or replacement of sensors from the sensor array (60), restocking of disposable materials, additional cleaning and sanitization of areas in an operating room affected by an aberration, and other maintenance tasks.

As another example, data collected by the plurality of ORMS (50) may be used to automatically generate and provide (512) daily reports to one or more recipients. Daily reports may be tailored for particular recipients and may include, for example, patient-specific reports, surgeon-specific reports, reports specific to other personnel or teams of personnel, room-specific reports, reports specific to a particular set of rooms, and other collections, sets, subsets, and analyses of data gathered by the sensor array (60) and/or displayed on various interfaces by individual ORMS (50).

As another example, data collected by the plurality of ORMS (50) may be used to update dynamic processing engines, such as may be included in some implementations of the room analysis engine (56). This may include aggregating sensor data and image data gathered from operating rooms, manual training inputs related to machine vision identification, physiological data gathered from patients during and after a procedure (e.g., such as data from post-operation follow-ups to diagnose infection and recovery), data gathered from the HIS (90) describing notes and determinations made by surgeons and other personnel following a procedure, and other information. Such information may be processed by the processor (100), the monitoring server (80), or both in order to update a machine learning process, machine vision process, or other dynamic or predictive process or dataset used by the room analysis engine (56). As one example, the processor (100) or HIS (90) may aggregate various datasets associated with the ORMS (50), anonymize or otherwise sanitize data as may be desired, and then submit such data to the monitoring server (80). The monitoring server (80) may process and incorporate some or all of the received data into a dataset (e.g., a universal dataset shared between all ORMS (50) systems, multiple ORMS (50) systems operated or served by a particular vendor, a customer-specific dataset shared between all ORMS (50) of a specific hospital, etc.), which may then be redistributed in updated form to one or more client ORMS (50) systems.

As another example, data collected by the plurality of ORMS (50) may be used to generate (516) case summaries for various aspects of the procedures associated with an ORMS (50). This may include generating (516) case summaries for specific patients, surgeons, or procedures, generalized case summaries related to higher-level topics such as infection, infection-reduction protocol adherence, EQI, and other topics. Generated (516) case summaries may be automatically provided to various parties (e.g., data scientists, risk assessors, insurance providers) as electronic communications, as will be apparent to those of ordinary skill in the art in light of this disclosure.

As another example, data collected by the plurality of ORMS (50) may be used to generate (518) various other analytics datasets beyond those already described. This may include, for example, analyzing several months of data from a plurality of ORMS (50) and automatically identifying certain guidelines from an infection-reduction protocol that are most likely to result in an infection when violated. Other analytic uses of the datasets provided by the ORMS (50) exist and will be apparent to those of ordinary skill in the art in light of this disclosure.

It shall be appreciated that various aspects of the present invention may be applicable to settings other than an operating room, including many other medical care rooms or facilities such as procedure rooms, where precise environmental control and adherence to a set of rules and procedures can enhance the likelihood of more positive medical outcomes, and that the scope of the present invention shall not be solely limited to the application to operating rooms and procedure rooms. Thus, in this document, “operating room” should be interpreted to mean any medical care room, facility, or other area where precise environmental control and adherence to a set of rules and procedures can enhance the likelihood of more positive medical outcomes.

The use of the terms “a” and “an” and “the” and similar references in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any unclaimed element as essential to the practice of the invention.

When an act is described herein as occurring “as a function of” or “based on” a particular thing, the system is configured so that the act is performed in different ways when one or more characteristics of the thing are different.

When something is characterized as “human-perceptible,” that thing is configured to be perceived by the mind or senses one or more relevant persons, as will occur to those skilled in the art.

While the invention has been illustrated and described in detail in the drawings and foregoing description, the same is to be considered as illustrative and not restrictive in character, it being understood that only the preferred embodiment has been shown and described and that all equivalents, changes, and modifications that come within the spirit of the inventions as described herein and/or by the following claims are desired to be protected. Hence, the proper scope of the present invention should be determined only by the broadest interpretation of the claims so as to encompass all such modifications as well as all relationships equivalent to those illustrated in the drawings and described in the specification.

What is claimed is:

1. An operating room monitoring system (50) for use, at least in part, within an operating room (10) to reduce the risks of infections, comprising:
a sensor array (60) configured to detect one or more events and conditions that affect a risk of infection in the operating room (10) and to produce signals corresponding to the events and conditions;
a monitoring server (80) configured to receive the signals produced by the sensor array (60) and compare the corresponding events and conditions with an infection-reduction protocol associated with the operating room; and
an indicator (106, 58) in the operating room, the indicator (106, 58) being operable to issue a human-perceptible indication as a function of a degree to which the events and conditions are determined to be compliant with the infection-reduction protocol.

2. The operating room monitoring system (50) of claim 1, wherein:
the sensor array (60) comprises an object recognizer configured to identify particular objects in the operating room; and
the comparing by the monitoring server (80) operates as a function of an output of the object recognizer.

3. The operating room monitoring system (50) of claim 2, wherein:
the object recognizer implements a predictive modeling engine,
the object recognizer distinguishes between foreground objects and background objects, and
an event identifier identifies particular events involving the foreground objects.

4. The operating room monitoring system (50) of claim 3, wherein the foreground objects include medical equipment.

5. The operating room monitoring system (50) of claim 3, wherein the infection-reduction protocol specifies one or more zones in the operating room where foreground objects are not to be located.

6. The operating room monitoring system (50) of claim 3, wherein the infection-reduction protocol distinguishes whether a human is properly wearing a mask.

7. The operating room monitoring system (50) of claim 1:
wherein the signals produced by the sensor array (60) comprise an environmental condition signal that characterizes the present environmental conditions in the operating room; and
further comprising an environmental monitor configured to receive the environmental condition signal and to issue an indication via the indicator (106, 58) when at least a portion of the environmental conditions in the operating room fail to comply with the infection-reduction protocol.

8. The operating room monitoring system (50) of claim 7, wherein:
the sensor array (60) comprises a temperature sensor (70) that produces a measurement of temperature in the operating room (10) and a humidity sensor (72) that produces a measurement of humidity in the operating room (10);
the environmental condition signal indicates the measurement of temperature and the measurement of humidity.

9. The operating room monitoring system (50) of claim 7, wherein
the sensor array (60) comprises an air pressure sensor that produces a measurement of air pressure at a point in the operating room (10); and
the environmental condition signal indicates the air pressure measurement.

10. The operating room monitoring system (50) of claim 7, wherein:
the sensor array (60) further comprises an air velocity sensor (66) that produces a measurement of airflow at a point in the operating room (10); and
the environmental condition signal further indicates the measurement of air flow.

11. The operating room monitoring system (50) of claim 7, wherein the sensor array (60) further comprises a door position sensor (68).

12. The operating room monitoring system (50) of claim 1, wherein the indicator (106, 58) presents a quality gauge (300) configured to provide, based upon data from the sensor array (60), information relating to air quality within one or more portions of the operating room (10).

13. The operating room monitoring system (50) of claim 1, wherein the indicator (106, 58) presents exception information in association with each indication, where the exception information identifies a rule in the infection-reduction protocol that was violated because of the particular event.

14. The operating room monitoring system (50) of claim 1, wherein the infection-reduction protocol is a function of information retrieved from an electronic medical record for a patient scheduled to be operated upon in the operating room (10).

15. The operating room monitoring system (50) of claim 1, wherein the identifying of the particular event or condition is triggered as a function of a signal from a particular sensor in the sensor array (60), where the particular sensor is a sensor that does not capture video.

16. The operating room monitoring system (50) of claim 15, wherein the particular sensor is selected from a group consisting of a proximity sensor (64), a door position sensor (68), an RFID reader, and a BLUETOOTH device.

17. An operating room monitoring system (50), comprising:
a processor (100) and a memory (104);
a sensor array (60) in communication with the processor (100) and memory (104), wherein each sensor of the sensor array (60) detects one or more conditions, events, or information within an operating room (10);
a display (21);
wherein the memory (104) is configured to store a protocol dataset that defines a set of rules as part of an infection-reduction protocol; and wherein the processor (100) is configured to:
during a procedure performed in the operating room (10), receive a procedure dataset from the sensor array (60), wherein the procedure dataset comprises data produced by at least one sensor in the sensor array (60);
display a dashboard interface (58) via the display (21) based on the procedure dataset;
generate a risk picture for the operating room (10) based on the procedure dataset, wherein the risk picture characterizes the risk of contamination in one or more areas of the operating room (10);
based on the procedure dataset, identify a protocol violation of the infection-reduction protocol; and
in response to the protocol violation, perform a response action selected from the response group consisting of:
providing a human-perceptible notification of the protocol violation;
updating the risk picture based on the protocol violation; and
updating and re-displaying the dashboard interface (58) based on the protocol violation.

18. The operating room monitoring system (50) of claim 17, wherein the sensor array (60) comprises three or more of:
a temperature sensor (70);
a humidity sensor (72);
an air pressure sensor;
an airflow sensor;
a door opening sensor;
an air velocity sensor (66);
a door position sensor (68);
an occupancy sensor;
an occupant position detector;
a carbon dioxide sensor
and infrared beam trip counter;
a camera (62);
a proximity sensor (64);
a near-real-time bio aerosol pathogen detector;
an RFID transceiver;
a video sensor, comprising
a video capture device that produces a video stream, and
a video processor that detects one or more conditions, events, or information within the operating room based on the video stream;
a particle count sensor; and
a microbial count sensor.

19. The operating room monitoring system (50) of claim 17, wherein the dashboard interface (58) displays three or more of:
an environmental quality index gauge (300),
a door status (306),
a door activity indicator (304),
a zone traffic indicator (302),
a dew point temperature indicator (308),
a room temperature indicator (310),
a room pressure indicator (312),
a room humidity indicator (314), and
a particle count indicator (316).

20. The operating room monitoring system (50) of claim 17, wherein the sensor array (60) comprises a set of one or more cameras (62), a set of one or more door position sensors (68), a set of one or more proximity sensors (64), and a set of one or more air velocity sensors (66).

21. The operating room monitoring system (50) of claim 20, wherein:
the set of cameras (62) are positioned within the operating room (10) to provide, in the aggregate, substantially unobstructed field of view of the operating room (10),
a door position sensor (68) in the set of door position sensors (68) is positioned at each door (32) of the operating room (10), and
each air velocity sensor (66) in the set of air velocity sensors (66) is positioned within a flow path of artificially circulated air within the operating room (10).

22. The operating room monitoring system (50) of claim 17, wherein:
each identifier of a set of identifiers is placed on a piece of medical equipment in the operating room (10), and
the sensor array (60) comprises a set of locator sensors, wherein the set of locator sensors is collectively operable to produce data indicating the position of each of the set of locators within the operating room.

23. The operating room monitoring system (50) of claim 17, wherein the processor (100) is further configured to:
store an operating room dataset that indicates:
ideal positions for each of a plurality of pieces of medical equipment within the operating room,
ideal positions for each of one or more personnel within the operating room, and
positions of a set of static features of the operating room, and
generate the risk picture (51) based on the operating room dataset.

24. The operating room monitoring system (50) of claim 23, wherein the processor (100) is further configured to provide a human-perceptible sensor placement guide based on the set of static features, wherein the sensor placement guide indicates an ideal position for each sensor of the sensor array (60) within the operating room (10).

25. The operating room monitoring system (50) of claim 17, wherein the dashboard interface (58) displays the risk picture (51) and an air change rate.

26. The operating room monitoring system (50) of claim 17, wherein:
the sensor array comprises one or more proximity sensors (64) that collectively produce a set of proximity data; and
the processor (100) is further configured to, when identifying one or more protocol violations, evaluate the set of proximity data in view of the set of rules to determine whether a risk zone incursion has occurred.

27. The operating room monitoring system (50) of claim 17, wherein:
the sensor array (60) comprises one or more cameras (62) that together produce a set of image data; and
the processor (100) is further configured to, when identifying one or more protocol violations:
perform a machine vision process on the set of image data to identify a set of objects within the operating room (10), and
evaluate positions of the set of objects based on the set of rules to determine whether a violation of the infection-reduction protocol has occurred.

28. The operating room monitoring system (50) of claim 17, wherein:
the sensor array (60) comprises one or more air velocity sensors (66) that together produce a set of air flow data;
the sensor array (60) further comprises one or more environmental sensors, the environmental sensors comprising at least one of a temperature sensor (70), a humidity sensor (72), a particle counter, and a microbial detector, and the environmental sensors producing a set of environmental quality data; and
the processor (100) is further configured to, when identifying one or more protocol violations:
evaluate the set of air flow data as a function of the set of rules to determine whether airflow is insufficient, and
evaluate the set of environmental quality data to determine whether an environmental quality index is insufficient.

29. The operating room monitoring system (50) of claim 17, wherein:
the sensor array (60) comprises one or more door position sensors (68) that produce a set of door position data; and
the processor (100) is further configured to, when identifying one or more protocol violations:
evaluate the set of door position data as a function of the set of rules to determine whether an excessive number of door openings has occurred, and
evaluate the set of door position data as a function of the set of rules to determine whether an excessive cumulative duration of door opening events has occurred.
